(19) United States
(12) Patent Application Publication
(10) Pub. No.: US 2024/0041753 A1
(43) Pub. Date: Feb. 8, 2024

(54) OIL BALM CLEANSING COSMETIC COMPOSITION

(71) Applicant: L V M H RECHERCHE, Saint-Jean-De-Braye (FR)

(72) Inventors: Yuri OKUTANI, Chiyoda-ku, Tokyo (JP); Mai OZAWA, Chiyoda-ku, Tokyo (JP)

(73) Assignee: L V M H RECHERCHE, Saint-Jean-De-Braye (FR)

(21) Appl. No.: 18/268,726

(22) PCT Filed: Dec. 22, 2020
(86) PCT No.: PCT/IB2020/001130

§ 371 Date: Jun. 21, 2023

(51) Int. Cl. A61K 8/92; A61Q 1/14; A61K 8/73; A61K 8/44; A61K 8/37

(52) U.S. Cl. CPC A61K 8/922; A61Q 1/14; A61K 8/73; A61K 8/442; A61K 8/37; A61K 2800/594

(57) ABSTRACT

The invention provides an oil balm cleansing cosmetic composition comprising (A) an oil and (B) an oil gelling agent, wherein component (B) comprises (B1) a polysaccharide fatty acid ester that includes a fatty acid ester of inulin, and (B2) a multichain multi-hydrophilic group-containing compound having a structure in which two N-acylamino acid salt molecules are linked with lysine.

### TECHNICAL FIELD

The present invention relates to an oil balm cleansing cosmetic composition.

### BACKGROUND ART

Cleansing cosmetic compositions for removal of makeup include aqueous cleansing gels which work by the cleansing efficacy of surfactants, emulsion type cleansing cosmetic compositions in the form of creams, and cleansing oils that utilize the dissolving oil components in makeup. Three types of cleansing oils are used, oil types (liquids), gel types (gels) and oil balm types (hard gels), in order of lower viscosity.

With cleansing cosmetic compositions it has been difficult to achieve both strength of cleansing efficacy and lack of oil residue on the skin after rinse-off, but cleansing oil gel are known which are said to be designed to solve this problem by comprising difatty acid lysine acylglutamate salts, which exhibit an ability to gel (Japanese Unexamined Patent Publication No. 2013-1698).

### Technical Problem

However, the present inventors have found that when electrolytes are added to gelatinous oily cleansing cosmetic compositions using difatty acid lysine acylglutamate salts, the oils are exudated from the gel, often producing stability issues such as phase separation or other phenomena.

It is common for currently used cosmetics to have added electrolytes such as preservatives, coloring agents and active ingredients, but addition of such electrolytes to compositions comprising difatty acid lysine acylglutamate salts has not allowed long- term stability to be maintained, and this has constituted a hindrance against their practical use.

So, it is an object of the invention to provide an oily cleansing cosmetic composition that not only has excellent cleansing properties, but even when electrolytes have been added, exhibits excellent stability as well as good spreadability and also excellent blend-ability with makeup.

### Solution to Problem and Summary of the Invention

The present invention provides an oil balm cleansing cosmetic composition comprising (A) an oil and (B) an oil gelling agent, wherein component (B) comprises (B1) a polysaccharide fatty acid ester that includes a fatty acid ester of inulin, and (B2) a multichain multi-hydrophilic group-containing compound having a structure in which two N-acylamino acid salt molecules are linked with lysine.

The oil balm cleansing cosmetic composition of the invention having the composition described above thus allows the oil content to be increased and exhibits high cleansing properties for makeup such as waterproof mascara. The combination of component (B1) and component (B2) also exhibits good stability even when electrolytes have been added.

The oil balm cleansing cosmetic composition of the invention also has good spreadability and excellent blend-ability with makeup.

The effects of a fresh sensation without greasiness is also exhibited. The blend-ability of cleansing cosmetic compositions during massage is an important property for evaluating their cleansing efficacy.

The oil balm cleansing cosmetic composition of the invention may further comprise (C) one or more optional ingredients selected from the group consisting of preservatives, humectants, coloring agents and active ingredients and one or more of the optional ingredients may be electrolytes.

The optional ingredients are ingredients that are suitable for addition to products, and since the long-term stability is maintained even by their addition, it becomes possible to provide an oil balm cleansing cosmetic composition suitable for practical use. Since a variety of components can also be added, this widens the range of application and allows various products to be designed to match consumer preferences.

The polysaccharide fatty acid ester (B1) may further include a straight-chain fatty acid ester of dextrin. The polysaccharide fatty acid ester is also preferably an ester of a polysaccharide and one or more saturated fatty acids of 12 to 22 carbon atoms.

Since it is not possible to ensure stability (especially high temperature stability) when a branched fatty acid ester is used as a fatty acid ester of dextrin, it is necessary to use a straight-chain fatty acid ester instead. Component (B 1) may be classified as a surfactant in some cases, but according to the invention, component (B 1) is considered to be an oil gelling agent and is treated differently from a normal surfactant.

The multichain multi-hydrophilic group-containing compound (B2) is preferably a compound represented by the following formula (1).

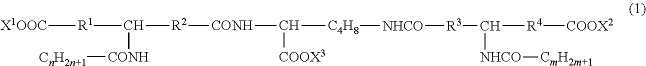

wherein
X¹, X² and X³ each independently represent an alkali metal atom, one of R¹ and R² is a single bond while the other is a methylene or ethylene group,
one of R³ and R⁴ is a single bond while the other is a methylene or ethylene group, and
n and m each independently represent an integer of 11 to 21.

Since, among the combinations of (R¹ and R²) and (R³ and R⁴), one is a single bond while the other is a methylene or ethylene group, the compound (B2) represented by formula (1) has a structure in which an N-acylglutamic acid salt or N-acylaspartic acid salt is linked by lysine.

In such a structure, by combination of the oil gelling agent having a structure in which amino acid-type surfactants are linked with spacers (lysine residues) (also known as a gemini type structure) and component (B1), properties desired for a cleansing agent are exhibited, such properties including not only preventing exudation of the oil but also excellent spreadability and a fresh sensation without greasiness. Component (B2) may be classified as a surfactant in some cases, but according to the invention, component (B2) is considered to be an oil gelling agent and is treated differently from a normal surfactant.

A humectant, as one type of the optional ingredient (C), may contain a polyol. However, since using a diol for the invention may potentially hamper gel formation, the polyol used is preferably a polyol with 3 or more hydroxyl groups in the molecule. In other words, using a polyol with 3 or more hydroxyl groups in the molecule easily provides sufficiently high viscosity as an oil balm and also exhibits moisturizing effect as the originally intended function.

The present invention also provides a cosmetic process for cleansing and/or caring keratinic materials, comprising the application onto keratinic materials, in particular onto skin, of the oil balm cleansing.

The present invention further provides a cosmetic process, wherein the oil balm cleansing provides to the keratinic cosmetic compositions on which it is applied, a high cleansing ability and good spreadability.

### ADVANTAGEOUS EFFECTS OF INVENTION

According to the invention it is possible to provide an oil balm cleansing cosmetic composition that not only has excellent cleansing properties, but even when electrolytes have been added, exhibits excellent stability as well as good spreadability and also excellent blend-ability-with makeup.

### DETAILED DESCRIPTION OF THE INVENTION

The oil balm cleansing cosmetic composition corresponds to an oiliness cleansing agent that is of an oil balm type. An “oil balm type” means a composition having a viscosity of 50,000 cps (50 Pa·s) or greater at 25° C. The viscosity is in particular measured by using a B-type rotary viscometer (for example, Rheolab QC manufactured by Anton Parr Co., Ltd.), and an ancre type spindle is inserted into an oil balm cleansing cosmetic composition held at 25° C. in a vessel having an inner diameter of 4.5 cm, and the rotation speed is measured at 10 rpm. In a preferred embodiment, the viscosity of the composition at 25° C. ranges from 50,000 to 150,000 cps.

### Cleansing Oil (A)

The oil balm cleansing cosmetic composition contains an oil (A), and any oil that can be used as a cleansing agent for cosmetics may be used as component (A).

In a particular embodiment, the cleansing oil (A) is selected from the group consisting of a fatty oil-based cleansing oil, an ester oil- based cleansing oil, a mineral oil-based cleansing oil, and mixtures thereof. Fatty oil-based cleansing oils include olive oil, rapeseed oil, camellia oil, sunflower seed oil, macadamia nut oil, corn oil, rice bran oil, avocado oil and meadowfoam seed oil, and ester oil-based cleansing oils include isononyl isononanonate, ethylhexyl palmitate, cetyl ethylhexanoate, isopropyl palmitate, isopropyl myristate, triethylhexanoin and tridecyl isononanoate. Mineral oil-based cleansing oils include mineral oils, isohexadecane and hydrogenated polyisobutene.

### Oil Gelling Agent (B)

The oil balm cleansing cosmetic composition also comprises an oil gelling agent (B) that comprises (B1) a polysaccharide fatty acid ester that includes a fatty acid ester of inulin and (B2) a multichain multi-hydrophilic group-containing compound having a structure in which two N-acylamino acid salt molecules are linked with lysine (B2).

### Fatty Acid Ester (B1)

Component (B1) has a fatty acid ester of inulin as an essential component, and so long as it contains this component it may also comprise a straight-chain fatty acid ester of dextrin. In other words, component (B1) may consist of a fatty acid ester of inulin alone, or it may comprise both a fatty acid ester of inulin and a straight-chain fatty acid ester of dextrin.

One of the important properties of an oil balm cleansing cosmetic composition is spreadability, i.e. a blend-ability with makeup, but this property is insufficient when using only a straight-chain fatty acid ester of dextrin as the polysaccharide fatty acid ester. Another important property for an oil balm cleansing cosmetic composition is a breaking sensation where the oil balm is transformed to an oil during massage. This property is slightly more superior with a straight-chain fatty acid ester of dextrin than with a fatty acid ester of inulin. In order to exhibit both spreadability and a breaking sensation, therefore, it is preferred to use a combination of a fatty acid ester of inulin and a straight-chain fatty acid ester of dextrin, rather than a fatty acid ester of inulin alone. However, the cosmetic composition can be sufficiently used in practice even with a fatty acid ester of inulin alone.

When a fatty acid ester of inulin and a straight-chain fatty acid ester of dextrin are combined, preferably the latter is used at 50 to 500 parts by weight with respect to 100 parts by weight of the former, and more preferably the latter is used at 100 to 200 parts by weight with respect to 100 parts by weight of the former. In a particular embodiment, the component (B) is present in the composition in a total amount ranging from 0.5% to 5%, preferably from 1% to 4% by total weight of the composition. In component (B), component (B1) is preferably present at 0.5 to 2 wt% and component (B2) at 0.5 to 2 wt% by total weight of the composition.

The fatty acid ester of inulin may be an ester of inulin and one or more saturated fatty acids of 12 to 22 carbon atoms. The number of carbon atoms of the saturated fatty acid is preferably 12 to 18, and the saturated fatty acid is more preferably myristic acid with 14 carbon atoms, palmitic acid with 16 carbon atoms or stearic acid with 18 carbon atoms. The fatty acid ester of inulin is preferably a linear fatty acid ester of inulin.

Fatty acid esters of inulin can be represented by the following structural formula (2). In the formula, A is an acyl group of 12 to 22 carbon atoms, and multiple A groups may be the same or different. The letter q is an integer of 1 or greater. Some of the A groups may also be hydrogen atoms. Suitable groups for A include myristoyl having 14 carbon atoms, palmitoyl having 16 carbon atoms and stearoyl having 18 carbon atoms.

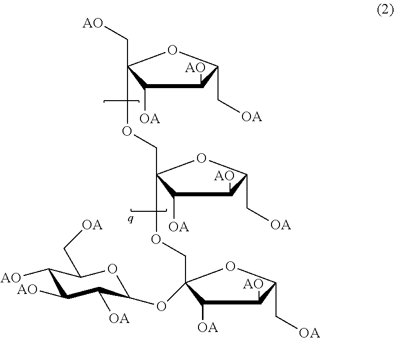

A fatty acid ester of dextrin may be an ester of dextrin and one or more saturated straight-chain fatty acids of 12 to 22 carbon atoms. The number of carbon atoms of the saturated straight-chain fatty acid is preferably 12 to 18, and the saturated fatty acid is more preferably myristic acid with 14 carbon atoms, palmitic acid with 16 carbon atoms or stearic acid with 18 carbon atoms.

Fatty acid esters of dextrin can be represented by the following structural formula (3a) or (3b). In the formulas, B is an acyl group of 12 to 22 carbon atoms, and multiple B groups may be the same or different. Some of the B groups may also be hydrogen atoms. Suitable groups for B include myristoyl having 14 carbon atoms, palmitoyl having 16 carbon atoms and stearoyl having 18 carbon atoms.

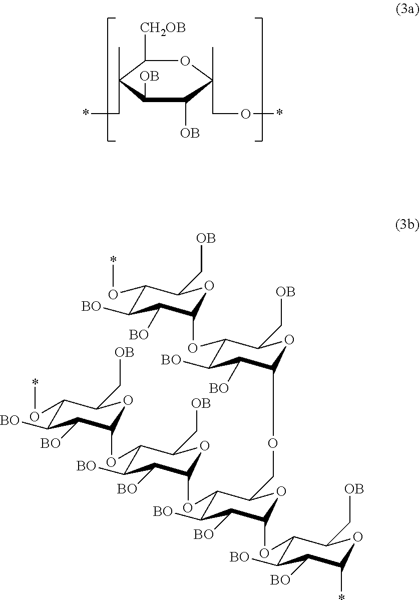

In a particular and preferred embodiment, the oil balm cleansing cosmetic composition comprises a stearoyl inulin alone or in combination with dextrin palmitate, as component (B1).

### Multichain Multi-Hydrophilic Group-Containing Compound (B2)

The multichain multi-hydrophilic group-containing compound having a structure in which two N-acylamino acid salt molecules are linked with lysine (B2), as a component other than component (B1) in the oil gelling agent (B) can be represented by the following formula (1), for example.

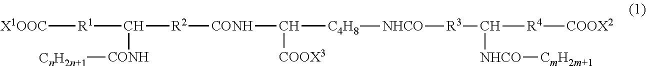

wherein X¹, X² and X³ are each independently an alkali metal atom,
one of R¹ and R² is a single bond while the other is a methylene or ethylene group,
one of R³ and R⁴ is a single bond while the other is a methylene or ethylene group, and
n and m each independently represent an integer of 11 to 21.
In particular embodiments, X¹, X² and X³ are each independently an alkali metal atom, with sodium and potassium atoms being preferred, and sodium atoms being more preferred. X¹, X² and X³ are preferably the same alkali metal atom. Preferably, n and m are each independently an integer of 11 to 17, with 13, 15 or 17 being more preferred. The letters n and m are preferably the same integer.

Compounds (B2) represented by formula (1) can be represented by formulas (1a) to (1d) below, for example. The definitions and preferred examples for X¹, X², X³ , n and m are the same as specified above. When X¹, X² and X³ are all sodium atoms and n and m are both 11, the compounds represented by formulas (1a) to (1d) correspond to sodium lysine dilauroylglutamate, that is a preferred component (B2).

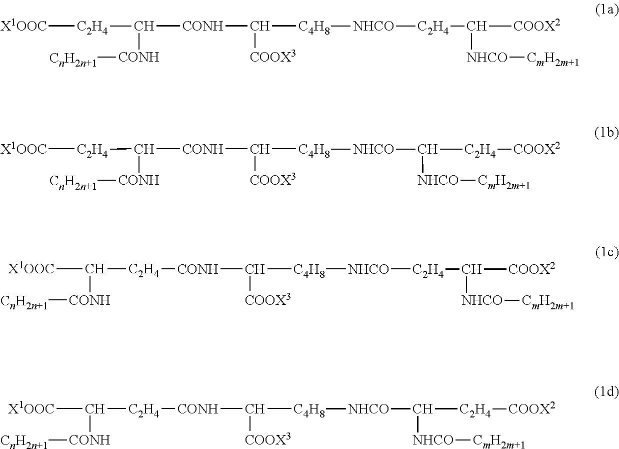

Compounds (B2) represented by formula (1) can also be represented by formulas (1e) to (1h) below. The definitions and preferred examples for X¹, X², X³, n and m are the same as specified above. When X¹, X² and X³ are all sodium atoms and n and m are both 11, the compounds represented by formulas (1e) to (1f correspond to sodium lysine dilauroylaspartate.

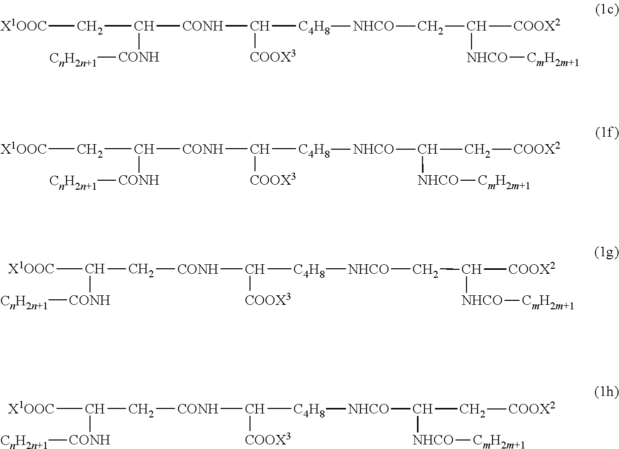

Component (B2) preferably comprises either or both a lysine dilauroylglutamate salt and a lysine dilauroylaspartate salt, and more preferably it comprises a lysine dilauroylglutamate salt (especially a sodium salt).

In a particular embodiment, the component (B2) is present in the composition in an amount ranging from 0.2 to 3%, preferably from 0.5 to 2% by total weight of the composition.
In a particular and preferred embodiment, the oil balm cleansing cosmetic composition comprises stearoyl inuline, dextrin palmitate and a sodium lysine dilauroylglutamate.

### Optional Ingredients (C)

The oil balm cleansing cosmetic composition may further comprise (C) one or more optional ingredients selected from the group consisting of preservatives, humectants, coloring agents and active ingredients, in which case at least one of them may be an electrolyte. The term “electrolyte” refers to a substance that ionizes into a cation and an anion when dissolved in a solvent, and according to the invention it is preferably a substance having an acid group such as carboxylic acid, sulfonic acid or phosphoric acid, or its salt. Salts include sodium salts, potassium salts, calcium salts, barium salts, strontium salts and ammonium salts.

Preservatives to be used as component (C) include antiseptics and antioxidants, specific examples of which are chlorhexidine digluconate, hydroxyacetophenone, ethylhexylglycerin, phenethyl alcohol, capryl glycol, sodium benzoate, isopropylmethylphenol, ethyl paraben, benzalkonium chloride, glyceryl caprylate, glycerin fatty acid esters, chlorphenesin, salicylic acid, potassium sorbate, sodium dehydroacetate, ethyl paraoxybenzoate, butyl paraoxybenzoate, propyl paraoxybenzoate, methyl paraoxybenzoate, bisabolol, hinokitiol, phenylethyl alcohol, phenethyl alcohol, phenoxyethanol, butylparaben, propylparaben, benzalkonium chloride, methylisothiazolinone, methylchloroisothiazolinone and methylparaben.

Humectants to be used as component (C) include glycerin, glucitol (sorbitol), octanediol, hexanediol, pentanediol, butylene glycol, polyethylene glycol, amino acids such as phenylalanine and leucine, amino acid salts such as histidine hydrochloride and lysine hydrochloride, saccharides, and plant extracts such as aloe extract. For increased gelling ability, the humectant used is preferably a polyol with 3 or more hydroxyl groups in the molecule, and more preferably it is a polyol with 3 to 6 hydroxyl groups in the molecule.

A coloring agent used as component (C) may be any inorganic pigment, organic synthetic dye or natural pigment. Organic synthetic dyes include FD & C Red No. 4, FD & C Yellow No. 5, FD & C Yellow No. 6, FD & C Blue No. 1 and FD & C Blue No. 2.

Active ingredients to be used as component (C) include components with beautifying effects on the skin, including effects against skin roughening or wrinkles, for example. Examples of such active ingredients include fat-soluble vitamins, and specifically vitamin A compounds such as retinol, retinol palmitate and retinol acetate, vitamin D compounds such as ergocalciferol and cholecalciferol, and vitamin E compounds such as tocopherols and tocotrienols, as well as dl-camphor, l-menthol, glycyrrhizinic acid, dipotassium glycyrrhizinate, salicylic acid, urea and hinokitiol. Active ingredients may also function as pigments, as is the case with carotenoids such as astaxanthin, lycopene and fucoxanthin.

These components to be used as component (C) are all suitable for addition to oil balm cleansing cosmetic compositions of the invention, and even if the components qualify as electrolytes, exudation of oil is still prevented while maintaining adequate cleansing properties and spreadability.

Component (C) may also comprise components other than preservatives, humectants, coloring agents and active ingredients, such as surfactants (anionic surfactants, cationic surfactants or nonionic surfactants), perfumes, water and solvents.

In a particular embodiment, the content of component (A) in the oil balm cleansing cosmetic composition ranges from 60 to 90wt %, preferably from 70 to 90 wt % and the content of component (B) ranges from 0,5 to 5%, preferably from 1 to 4 wt %. In component (B), component (B1) is preferably present at 0.5 to 2 wt % and component (B2) at 0.5 to 2 wt %. The total content of component (C) may be 0.8 to 3.0 wt %.

The oil balm cleansing cosmetic composition comprising these optional ingredients can be produced by the following method, for example. Specifically, first component (B2), water, a preservative and a coloring agent are uniformly mixed while heating. Separately, component (A), component (B1) and a surfactant are uniformly mixed while heating. Both mixtures are uniformly mixed together at a predetermined mixing rate, and finally a perfume is added.

The oil balm cleansing cosmetic composition may be filled into a sealed container such as a tube, bottle or can. Storage is preferably indoors or in a cool dark space.

### Examples

The present invention will now be explained by the following examples, with the understanding that the invention is not limited by the examples.

Phase 1 listed in Table 1 was mixed at 40° C. until the bulk reached homogeneity. The cleansing agent (oil) of Phase 2 was heated to 80° C., dispersing the surfactant and oil gelling agent, and the dispersion was mixed until the bulk reached homogeneity. Phase 2 was added to Phase 1 at 5%/min, and the mixture was mixed for 20 minutes after addition of Phase 2. After confirming that the bulk was a homogeneous gel, cooling to room temperature was initiated. Phase 3 was then added and mixed until the bulk reached homogeneity, to obtain oil balm cleansing cosmetic compositions for the Examples and Comparative Examples. Electrolytes were included in the oil balm cleansing cosmetic compositions of the Examples and Comparative Examples.

TABLE 1

| | Chemical | | Example | Example | Comp. | Comp. | Comp. | Comp. | Comp. | Comp. |
|---|---|---|---|---|---|---|---|---|---|---|
| Phase | Description | Function | 1 | 2 | Example 1 | Example 2 | Example 3 | Example 4 | Example 5 | Example 6 |
| 1 | SODIUM | GELATION | 1.5 | 1.5 | 1.5 | 1.5 | 1.2 | 1.2 | 1.5 | 1.2 |
| | DILAURAMIDO- | AGENT | | | | | | | | |
| | GLUTAMIDE | | | | | | | | | |
| | LYSINE AND | | | | | | | | | |
| | WATER | | | | | | | | | |
| 1 | GLYCERIN | HUMECTANT | 16.7 | 16.7 | 16.7 | 18.03 | 17 | 17 | 16.7 | 17 |
| 1 | D-GLUCITOL | HUMECTANT | 5 | 5 | 5 | 5 | 5 | 5 | 5 | 5 |
| 1 | AQUA | | 1.38 | 1.38 | 1.38 | 2.75 | 1.38 | 1.38 | 1.38 | 1.38 |
| 1 | CHLORHEXIDINE | PRESERVATIVE | | 0.22 | 0.22 | 0.22 | 0.22 | 0.22 | 0.22 | 0.22 |
| | DIGLUCONATE | | | | | | | | | |
| 1 | HYDROXYACE- | PRESERVATIVE | 0.3 | 0.3 | 0.3 | 0.3 | 0.3 | 0.3 | 0.3 | 0.3 |
| | TOPHENONE | | | | | | | | | |
| 1 | ETHYLHEXYL | PRESERVATIVE | 0.5 | 0.5 | 0.5 | 0.5 | 0.5 | 0.5 | 0.5 | 0.5 |
| | GLYCERIN | | | | | | | | | |
| 1 | PHENETHYL | PRESERVATIVE | 0.3 | 0.3 | 0.3 | 0.3 | 0.3 | 0.3 | 0.3 | 0.3 |
| | ALCOHOL | | | | | | | | | |
| 1 | Caprylyl Glycol | PRESERVATIVE | — | — | — | 0.3 | — | — | — | — |
| 1 | COLORANT | COLORANT | 0.1 | 0.1 | 0.1 | 0.1 | 0.1 | 0.1 | 0.1 | 0.1 |
| 2 | ETHYLHEXYL | CLEANSING | 48 | 48 | 48 | 44 | 48.3 | 49 | 48 | 49 |
| | PALMITATE | AGENT | | | | | | | | |
| 2 | ISONONYL | CLEANSING | 16.6 | 16.6 | 16.6 | 16.6 | 16.6 | 16.6 | 16.6 | 16.6 |
| | ISONONANOATE | AGENT | | | | | | | | |
| 2 | UNDECANE/ | CLEANSING | 5 | 5 | 5 | 5 | 5 | 5 | 5 | 5 |
| | TRIDECANE/ | AGENT | | | | | | | | |
| | TOCOPHEROL/ | | | | | | | | | |
| | HELIANTHUS | | | | | | | | | |
| | ANNUUS (SUNFLOWER) | | | | | | | | | |
| | SEED OIL | | | | | | | | | |
| 2 | PEG-20 GLYCERYL | SURFACTANT | 2 | 2 | 2 | 2 | 2 | 2 | 2 | 2 |
| | TRIISOSTEARATE | | | | | | | | | |
| 2 | GLYCERYL STEARATE | SURFACTANT | — | — | — | — | — | 1 | — | — |
| | | (HLB: 3) | | | | | | | | |
| 2 | SORBITAN | SURFACTANT | 1 | 1 | 1 | 1 | 1 | — | 1 | — |
| | MONOSTEARATE | (HLB: 4.7) | | | | | | | | |
| 2 | PEG-7 GLYCERYL | SURFACTANT | — | — | — | — | — | — | — | 1 |
| | COCOATE | (HLB: 13) | | | | | | | | |
| 2 | DEXTRIN PALMITATE | GELATION | — | 0.5 | 1 | 2 | 0.7 | — | — | — |
| | | AGENT | | | | | | | | |
| 2 | STEAROYL INULIN | GELATION | 1 | 0.5 | — | — | — | — | — | — |
| | | AGENT | | | | | | | | |
| 2 | DEXTRIN | GELATION | — | — | — | — | — | — | 1 | — |
| | PALMITATE/ | AGENT | | | | | | | | |
| | ETHYLHEXANOATE | | | | | | | | | |
| 3 | PERFUME | PERFUME | 0.4 | 0.4 | 0.4 | 0.4 | 0.4 | 0.4 | 0.4 | 0.4 |

The oil balm cleansing cosmetic compositions of the Examples and Comparative Examples were tested for pH, viscosity, stability and sensory evaluation, by the following methods. The results are summarized in Table 2.

### pH

The pH at 25° C. was measured with a pH meter F-51 manufactured by HORIBA, Ltd.

### Viscosity

Using a B-type rotary viscometer (Rheolab QC manufactured by Anton Parr Co., Ltd.), an ancre type spindle was inserted into an oil balm cleansing cosmetic composition held at 25° C. in a vessel having an inner diameter of 4.5 mm and the viscosity was measured at a rotation speed of 10 rpm.

### Stability

(1) Stability at 4° C., 25° C. (room temperature), 45° C. and 50° C. The oil balm cleansing cosmetic composition was filled into a transparent container, and after sealing the cover, it was stored for one month at 4° C., 25° C. (room temperature), 45° C. or 50° C. Following storage, it was restored to room temperature (25° C.) and oil exudation was observed. Observation was based on: stability without exudation of oil, visible exudation of oil, or occurrence of phase separation.
(2) Cycle test at 40° C. and −10° C. The oil balm cleansing cosmetic composition was filled into a transparent container, and after sealing the cover, a cycle of storage at 40° C. for 12 hours and storage at −10° C. for 12 hours was repeated, for a total of one month of storage. Following the cycle test, it was restored to room temperature (25° C.) and oil exudation was observed. Observation was based on: stability without exudation of oil, visible exudation of oil, or occurrence of phase separation.

### Evaluation of Cleansing Efficacy

The above-mentioned oil balm cleansing cosmetic composition was evaluated for cleansing efficacy against waterproof mascara by the following method. On a vinyl chloride plate, 5 mg of waterproof mascara (Mascara Diorshow Iconic Overcurl, Waterproof, product of Dior Corp.) was applied in a circular shape of 1 cm, and dried for more than 1 night. 20 mg of the oil balm cleansing cosmetic composition of Examples and Comparative Examples was applied to the mascara, and the mascara was massaged 80 times with fingers. The condition of remaining mascara after rinsing with tap water was compared to evaluate the cleansing ability.
A: Excellent
B: Slightly inferior
C: Inferior

### Sensory Evaluation of Spreadability

The spread-ability was evaluated by 10 cosmetic expert evaluation panels of the organization to which the present inventors belong.
A: Excellent
B: Slightly inferior
C: Inferior

TABLE 2

| | | | Comp. | Comp. | Comp. | Comp. | Comp. | Comp. |
|---|---|---|---|---|---|---|---|---|
| | Example 1 | Example 2 | Example 1 | Example 2 | Example 3 | Example 4 | Example 5 | Example 6 |
| pH | 7.18 | 7.27 | 7.10 | 7.16 | 7.20 | 6.86 | 7.15 | 7.11 |
| Viscosity (cps) | 76220 | 112700 | 135400 | 72450 | 98230 | 95100 | 130300 | 115900 |
| Stability | | | | | | | | |
| 4 C., 1 month | Stable | Stable | Stable | Stable | Stable | Stable | Oily | Oily |
| | | | | | | | exudation | exudation |
| 45 C., 1 month | Stable | Stable | Stable | Stable | Stable | Stable | Separation | Separation |
| 50 C., 1 month | Stable | Stable | Stable | Stable | Stable | Oily | Separation | Separation |
| | | | | | | exudation | | |
| Cycle, 1 month | Stable | Stable | Stable | Stable | Stable | Separation | Separation | Separation |
| Room temperature | Stable | Stable | Stable | Stable | Stable | Stable | Stable | Separation |
| Cleansing ability | A | A | A | A | A | — | — | — |
| Spreadability | A | A | B | B | B | — | — | — |

These results demonstrate that the combination of (A) an oil and (B) an oil gelling agent, wherein component (B) comprises (B1) a polysaccharide fatty acid ester that includes a fatty acid ester of inulin, and (B2) a multichain multi-hydrophilic group-containing compound having a structure in which two N-acylamino acid salt molecules are linked with lysine, provides the better performance of cleansing ability and spreadability, in comparison with comparative examples. In particular, the comparative example 1 illustrates that the spreadability, i.e. a blend-ability with makeup, is insufficient when using only a straight-chain fatty acid ester of dextrin as the polysaccharide fatty acid ester.

1. An oil balm cleansing cosmetic composition comprising (A) an oil and (B) an oil gelling agent, wherein component (B) comprises (B1) a polysaccharide fatty acid ester that includes a fatty acid ester of inulin, and (B2) a multichain multi-hydrophilic group-containing compound having a structure in which two N-acylamino acid salt molecules are linked with lysine.

2. The oil balm cleansing cosmetic composition according to claim 1, which further comprises (C) one or more optional ingredients selected from the group consisting of preservatives, humectants, coloring agents and active ingredients, wherein one or more of the optional ingredients are electrolytes.

3. The oil balm cleansing cosmetic composition according to claim 1, wherein the polysaccharide fatty acid ester (B1) comprises a fatty acid ester of inulin and a straight-chain fatty acid ester of dextrin.

4. The oil balm cleansing cosmetic composition according to claim 1, wherein the polysaccharide fatty acid ester (B1) is an ester of a polysaccharide and one or more saturated fatty acids of 12 to 22 carbon atoms.

5. The oil balm cleansing cosmetic composition according to claim 1, wherein the multichain multi-hydrophilic group-containing compound (B2) is a compound represented by the following formula (1). 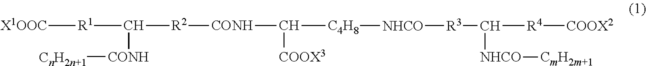
wherein
X¹, X², and X³ each independently represent an alkali metal atom,
one of R¹ and R² is a single bond while the other is a methylene or ethylene group,
one of R³ and R⁴ is a single bond while the other is a methylene or ethylene group, and
n and m each independently represent an integer of 11 to 21.

6. The oil balm cleansing cosmetic composition according to claim 2, wherein component (B) comprises a stearoyl inulin and dextrin palmitate and a sodium lysine dilauroylglutamate.

7. The oil balm cleansing cosmetic composition according to claim 2, wherein oil (A) is selected from the group consisting of fatty oil-based cleansing oil, an ester oil-based cleansing oil, a mineral oil-based cleansing oil, and mixtures thereof.

8. The oil balm cleansing cosmetic composition according to claim 2, wherein the total amount of oil(s) (A) ranges from 60% to 90%, by total weight of the composition.

9. The oil balm cleansing cosmetic composition according to claim 2, wherein the humectant comprises a polyol with 3 or more hydroxyl groups in the molecule.

10. A cosmetic process for cleansing and/or caring for keratinic materials, comprising the application onto keratinic materials, of the oil balm cleansing according to claim 1.

11. The cosmetic process according to claim 10, wherein the oil balm cleansing provides to the keratinic materials on which it is applied a high cleansing ability and good spreadability.

12. The cosmetic process according to claim 10, wherein the oil balm cleansing is applied onto skin.
